(19) United States
(12) Patent Application Publication
(10) Pub. No.: US 2026/0096918 A1
(43) Pub. Date: Apr. 9, 2026

(54) NON-WOVEN FABRIC MOUTH TAPE SUPPORTING NASAL BREATHING WITH INTEGRATED SILICONE GEL

(71) Applicant: Gina Everett Miller, Cary, NC (US)

(72) Inventor: Gina Everett Miller, Cary, NC (US)

(21) Appl. No.: 19/350,698

(22) Filed: Oct. 6, 2025

Related U.S. Application Data
(60) Provisional application No. 63/705,133, filed on Oct. 9, 2024.

(51) Int. Cl. A61F 5/56; A61F 5/37; B32B 5/02; B32B 7/06; B32B 7/12; B32B 25/10; B32B 25/20; D04H 1/4291; D04H 1/435; D04H 1/56

(52) U.S. Cl. CPC A61F 5/56; A61F 5/3707; B32B 5/022; B32B 7/06; B32B 7/12; B32B 25/10; B32B 25/20; D04H 1/4291; D04H 1/435; D04H 1/56; B32B 2262/0253; B32B 2262/0276; B32B 2307/724; D10B 2321/022; D10B 2331/04; D10B 2509/00

(57) ABSTRACT

A mouth tape for promoting nasal breathing during sleep comprises a non-woven fabric substrate with an adhesive surface for adhering to perioral skin, and an integrated silicone gel section with a gentle adhesive surface for contacting the lips. The silicone gel section is positioned within the non-woven fabric substrate, creating a configuration where sensitive lip tissue contacts only the gentle silicone gel while the stronger adhesive of the non-woven fabric provides secure attachment to less sensitive perioral skin. This design maintains mouth closure throughout sleep while allowing trauma-free removal upon waking. Included ventilation features enhance user comfort. The invention solves the long-standing problem of balancing secure adhesion with gentle removal in mouth tape applications.

### CROSS-REFERENCE TO RELATED APPLICATION

This application claims the benefit of U.S. Provisional Patent Application No. 63/705,133, filed Oct. 9, 2024, the entire disclosure of which is incorporated herein by reference.

### FIELD OF THE INVENTION

The present invention relates generally to sleep aids, and more particularly to mouth tapes configured to promote nasal breathing during sleep while providing enhanced comfort and protection to the lips and surrounding oral area.

### BACKGROUND OF THE INVENTION

Mouth breathing during sleep is associated with numerous health issues including snoring, sleep apnea, dry mouth, dental problems, and reduced sleep quality. To address these issues, mouth tapes have been developed to encourage nasal breathing by maintaining the mouth in a closed position during sleep.

Conventional mouth tapes typically consist of adhesive fabric that is applied directly across the lips. While these traditional tapes are effective at maintaining mouth closure throughout the night, they suffer from significant drawbacks. The adhesive used to secure conventional fabric tape is often harsh and aggressive, causing irritation, pain, and even injury to the delicate skin of the lips upon removal. This is particularly problematic given that users typically apply these products nightly, leading to cumulative damage over time.

In response to the comfort issues associated with traditional mouth tapes, some manufacturers have developed products using only silicone gel adhesives. While these silicone-only products are more comfortable to wear and remove, they suffer from inadequate adhesion strength. These products frequently fall off during sleep, completely negating their intended purpose of maintaining mouth closure.

The prior art fails to provide a solution that adequately balances the competing requirements of secure adhesion throughout the night and comfortable removal without tissue damage. No existing products successfully integrate the security benefits of non-woven adhesive fabric with the comfort benefits of silicone gel in a single, unified tape design.

### SUMMARY OF THE INVENTION

The present invention comprises a non-woven fabric substrate having an adhesive surface, with an integrated silicone gel section having an adhesive surface positioned within the non-woven fabric substrate.

In preferred embodiments, the silicone gel section is centrally located within the non-woven fabric substrate, creating a protective zone that contacts the most sensitive areas of the lips while the surrounding non-woven fabric border provides secure adhesion to the less sensitive perioral skin.

The invention may further include strategically placed vents in various components to allow for moisture release and reduced restriction during use. A removable release paper protects the adhesive surfaces prior to application.

The primary advantages of the present invention include: (1) secure adhesion throughout the night due to the non-woven fabric border; (2) protection and comfort for sensitive lip tissue through the integrated silicone gel center; (3) painless removal without tissue damage; and (4) suitability for nightly use without cumulative harm to oral tissues. Additional benefits may include potential anti-aging effects through prevention of perioral wrinkle formation during sleep.

These and other features, aspects and advantages of the present invention will become better understood with reference to the following drawings, description and claims.

### BRIEF DESCRIPTION OF THE DRAWINGS

FIG. 1 is a front perspective view of the non-woven fabric mouth tape of the present invention;

FIG. 2 is rear perspective view of the invention;

FIG. 3 is a front perspective exploded view of the invention;

FIG. 4 is a rear perspective exploded view of the invention; and

FIG. 5 depicts the mouth tape in use on a user, illustrating proper placement and function.

### DETAILED DESCRIPTION OF THE INVENTION

The following detailed description is of the best currently contemplated modes of carrying out exemplary embodiments of the invention. The description is not to be taken in a limiting sense but is made merely for the purpose of illustrating the general principles of the invention, since the scope of the invention is best defined by the appended claims.

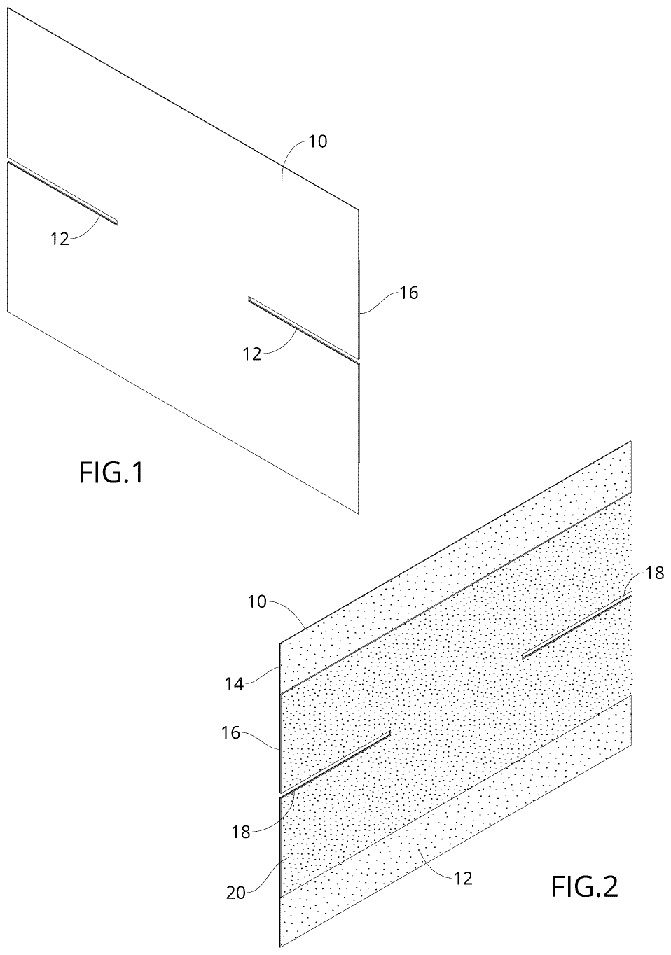

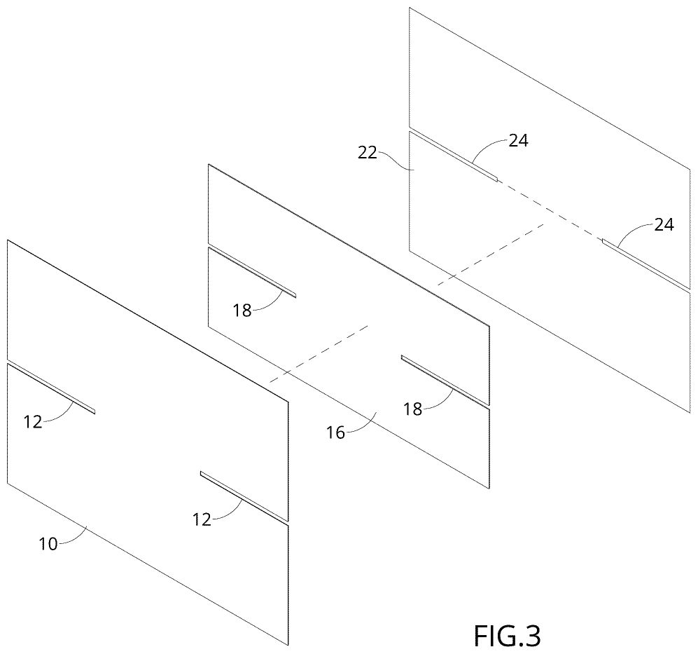

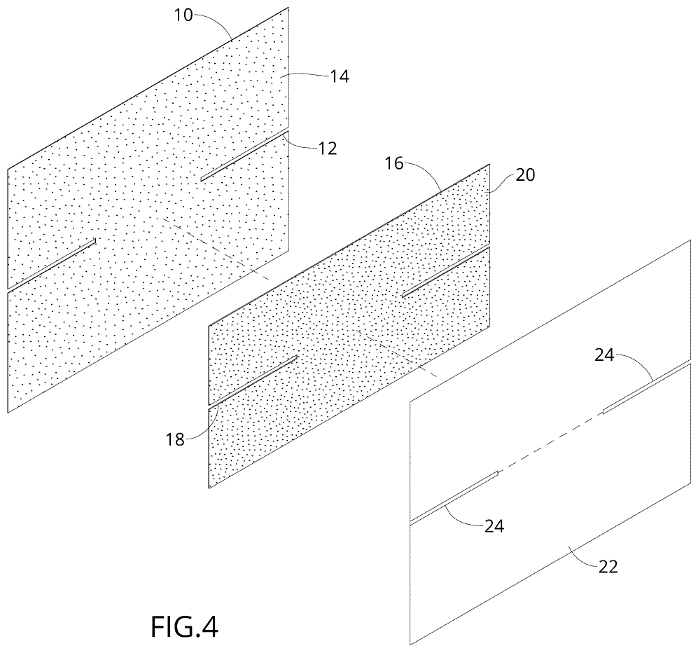

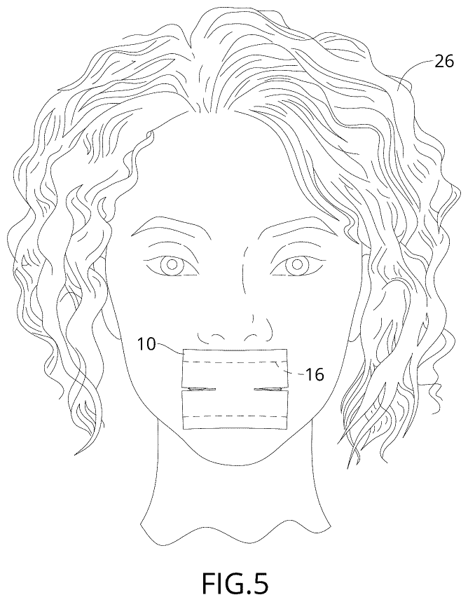

Referring now to FIGS. 1-5, wherein like numerals indicate like elements throughout the several views, the present invention will be described in detail.

### Overall Structure

As seen in FIGS. 1-5, the mouth tape of the present invention comprises three primary components: a non-woven fabric substrate (10), an integrated silicone gel section (16), and a removable release paper (22). These components work in concert to provide secure adhesion, user comfort, and protection of sensitive oral tissues.

### Non-Woven Fabric Substrate (10)

The non-woven fabric substrate (10) forms the primary structural element of the mouth tape. This substrate is preferably constructed from medical-grade, hypoallergenic non-woven fabric materials commonly used in medical adhesive applications. Suitable materials include, but are not limited to, spunbond polypropylene, spunlace polyester, or similar biocompatible non-woven fabrics.

The non-woven fabric substrate (10) includes an adhesive surface (14) configured to adhere to the user's skin. The adhesive used on surface (14) is preferably a medical-grade, skin-safe adhesive such as acrylate-based pressure-sensitive adhesives commonly used in medical tapes. The adhesive strength is selected to provide secure attachment to skin while allowing for removal without excessive trauma.

In preferred embodiments, the non-woven fabric substrate (10) includes strategically positioned vents (12). The silicone gel layer extends substantially across the entire length of the tape. The non-woven fabric layer is dimensionally larger than the gel layer, such that exposed fabric margins remain visible along opposing edges. The exposed margins represent a fraction of the overall tape width, while the gel layer spans the remaining majority portion. Horizontal vent openings are incorporated within the silicone gel layer. These vents are preferably configured as elongated openings that extend from the outer perimeter edges of the non-woven fabric substrate inwardly toward the central region. The vents (12) are arranged to terminate in the central area of the tape, creating pathways that facilitate airflow and moisture management while maintaining structural integrity of the adhesive bond.

The positioning of vents (12) extending from the outer edges toward the center provides several technical advantages: (1) creating controlled stress relief points that prevent tearing during removal; (2) allowing moisture vapor to escape from under the adhesive through defined channels; (3) providing tactile reference points for users during application and removal; and (4) maintaining adhesive contact in critical bonding areas while reducing overall restrictive sensation.

These vents serve multiple functions: (1) allowing moisture vapor transmission to prevent maceration of underlying skin; (2) providing slight pressure relief to enhance user comfort; and (3) facilitating easier removal by providing grip points for the user.

### Silicone Gel Section (16)

The silicone gel section (16) of the present invention is preferably constructed from medical-grade silicone gel materials known for their biocompatibility, gentle adhesion properties, and ability to protect sensitive tissues.

The silicone gel section (16) includes an adhesive surface (20) that contacts the user's lips. The same adhesive is applied to the non-woven fabric. The inherent properties of both the fabric and the gel provide for comfort and gentleness both during use and during removal of the device.

The silicone gel section (16) is preferably positioned centrally within the non-woven fabric substrate (10), creating a configuration where the silicone gel contacts the most sensitive central portion of the lips while the non-woven fabric border adheres to the less sensitive perioral skin.

Similar to the non-woven fabric substrate, the silicone gel section (16) may include vents (18) to provide moisture vapor transmission and enhanced user comfort.

### Positional Relationship

The silicone gel section (16) is positioned such that it will contact the user's lips when the tape is properly applied, while the surrounding border of non-woven fabric substrate (10) contacts the perioral skin.

The non-woven fabric, when combined with the adhesive, provides a stronger hold on surrounding, less sensitive skin. The same adhesive on the silicone gel layer will provide a gentler hold; however, is subject to detachment. This arrangement ensures that: (1) the most sensitive tissues (lips) contact only the gentle silicone gel; and (2) the combination provides both comfort and security throughout the intended period of use.

### Release Paper (22)

The release paper (22) serves as a protective covering for both adhesive surfaces (14, 20) prior to use. The release paper is preferably constructed from silicone-coated paper or film that can be easily removed while leaving the adhesive surfaces clean and ready for application.

The release paper (22) may include vents (24) that align with the vents in the underlying layers, facilitating manufacturing and potentially providing visual indicators for proper application.

### Ventilation System

The ventilation system comprising vents (12, 18, 24) provides several benefits. The vents allow for: (1) moisture vapor transmission to prevent skin maceration; (2) slight air exchange to reduce the sensation of restriction; and (3) easier handling and removal of the product.

The size, shape, and positioning of vents can be optimized based on user comfort requirements and manufacturing considerations. Vents may be circular holes, slits, or other shapes as appropriate for the specific application.

### Method of Use

Referring particularly to FIG. 5, the method of using the mouth tape will now be described. The user (26) first removes the release paper (22) to expose the adhesive surfaces (14, 20). With the mouth in a closed position, the user positions the silicone gel section (16) over the central portion of the lips, ensuring that the silicone gel adhesive surface (20) makes contact with the lip tissue.

The user then applies gentle pressure to secure the non-woven fabric substrate (10) to the perioral skin, beginning from the center and working outward to avoid air bubbles and ensure complete adhesion. The resulting configuration maintains the mouth in a closed position throughout sleep, promoting nasal breathing.

Upon waking, removal is accomplished by gently lifting an edge of the tape and slowly peeling it away from the skin and lips. The silicone gel section (16) releases gently from the lip tissue without causing trauma, while the non-woven fabric substrate (10) releases from the perioral skin with minimal discomfort.

### Manufacturing Process

The mouth tape is preferably manufactured using a multi-step process. The non-woven fabric substrate (10) is first prepared with its adhesive coating (14). Separately, the silicone gel section (16) is prepared with its adhesive surface (20). These components are then assembled, with the silicone gel section (16) being positioned and adhered to the appropriate location on the non-woven fabric substrate (10).

Any desired vents (12, 18) are created through cutting, punching, or molding processes as appropriate for the specific materials used. The release paper (22) is then applied over the entire adhesive surface, with vents (24) created as needed.

The assembled product is then cut to final dimensions and packaged for distribution.

### ALTERNATIVE EMBODIMENTS

While the preferred embodiment positions the silicone gel section (16) centrally within the non-woven fabric substrate (10), alternative configurations are contemplated. The silicone gel section could be repositioned, resized, or even divided into multiple sections based on specific user needs or anatomical considerations.

Similarly, the ventilation system can be modified or enhanced based on user preferences and specific applications. The overall dimensions of the mouth tape can be adjusted to accommodate different user populations or specific use cases.

The materials described herein represent preferred embodiments, but alternative biocompatible materials with similar properties could be substituted while maintaining the essential function of the invention.

### ADDITIONAL APPLICATIONS

While primarily designed for promoting nasal breathing during sleep, the mouth tape of the present invention may have additional applications. The gentle silicone gel section may help prevent or reduce perioral wrinkles by limiting facial muscle movement during sleep. The product may also be useful in medical settings where temporary mouth closure is desired.

It should be understood, of course, that the foregoing relates to exemplary embodiments of the invention and that modifications may be made without departing from the spirit and scope of the invention as set forth in the following claims.

What is claimed is:

1. A mouth tape for promoting nasal breathing, comprising:
a non-woven fabric substrate having a first adhesive surface configured to adhere to skin surrounding a user's mouth;
a silicone gel section having a second adhesive surface configured to contact the user's lips, wherein the silicone gel section is integrated with and positioned within the non-woven fabric substrate such that the silicone gel section is surrounded by a border of the non-woven fabric substrate; and
wherein the silicone gel section provides gentle adhesion to lip tissue while the non-woven fabric substrate provides secure adhesion to perioral skin.

2. The mouth tape of claim 1, wherein the silicone gel section is centrally positioned within the non-woven fabric substrate.

3. The mouth tape of claim 1, further comprising a removable release paper covering both the first adhesive surface and the second adhesive surface.

4. The mouth tape of claim 1, wherein the non-woven fabric substrate includes a plurality of vents extending from outer perimeter edges of the non-woven fabric substrate inwardly toward a central region of the mouth tape.

5. The mouth tape of claim 4, wherein the plurality of vents terminate in the central region.

6. The mouth tape of claim 1, wherein the silicone gel section includes at least one vent configured to allow moisture vapor transmission.

7. The mouth tape of claim 3, wherein the release paper includes at least one vent.

8. The mouth tape of claim 1, wherein the non-woven fabric substrate comprises a medical-grade, hypoallergenic material selected from the group consisting of spunbond polypropylene and spunlace polyester.

9. The mouth tape of claim 1, wherein the first adhesive surface comprises an acrylate-based pressure-sensitive adhesive.

10. The mouth tape of claim 1, wherein the silicone gel section comprises medical-grade silicone gel.

11. The mouth tape of claim 1, wherein the mouth tape is configured to maintain mouth closure during sleep while allowing trauma-free removal upon waking.

12. A method of promoting nasal breathing during sleep, comprising:
providing a mouth tape having a non-woven fabric substrate with a first adhesive surface and an integrated silicone gel section with a second adhesive surface;
positioning the silicone gel section over a central portion of a user's lips such that the second adhesive surface contacts the lip tissue;
securing the non-woven fabric substrate to perioral skin surrounding the user's mouth via the first adhesive surface; and
maintaining the mouth tape in position during sleep to encourage nasal breathing.

13. The method of claim 12, further comprising removing the mouth tape by gently peeling it away from the lips and perioral skin without causing tissue trauma.

14. The method of claim 12, wherein the silicone gel section protects sensitive lip tissue while the non-woven fabric substrate provides secure adhesion throughout a sleep period.
